(19) United States
(12) Patent Application Publication
(10) Pub. No.: US 2015/0056299 A1
(43) Pub. Date: Feb. 26, 2015

(54) FOOD SUPPLEMENT TO SUPPORT BRAIN FUNCTION

(71) Applicant: Venture Life Limited, Bracknell Berkshire (GB)

(72) Inventors: Jeremy Anthony Philip Randall, Bracknell Berkshire (GB); Mauro Podiffero, Bracknell Berkshire (GB)

(73) Assignee: VENTURE LIFE LIMITED, Bracknell Berkshire (GB)

(21) Appl. No.: 14/389,764

(22) PCT Filed: Apr. 2, 2013
(86) PCT No.: PCT/GB2013/050868

§ 371 Date: Sep. 30, 2014

(30) Foreign Application Priority Data
Apr. 2, 2012 (GB) 1205856.6

(51) Int. Cl. A61K 38/17; A61K 31/7072; A61K 31/7068; A61K 31/198; A61K 31/197; A61K 31/522; A61K 31/34; A61K 31/714; A61K 31/4415; A61K 31/202; A61K 31/661; A61K 36/16; A61K 36/185; A61K 31/519; A23L 1/305; A23L 1/302; A23L 1/304; A23L 1/30; A61K 33/26

(52) U.S. Cl. CPC A61K 38/1709; A61K 33/26; A61K 31/7072; A61K 31/7068; A61K 31/198; A61K 31/197; A61K 31/522; A61K 31/34; A61K 31/714; A61K 31/4415; A61K 31/202; A61K 31/661; A61K 36/16; A61K 36/185; A61K 31/519; A23L 1/3051; A23L 1/302; A23L 1/304; A23L 1/3002; A23L 1/30; A23V 2002/00
USPC 424/646

(57) ABSTRACT

The present invention relates to a food supplement to support brain function, including cognitive functions such as memory. The food supplement comprises one or more active ingredients selected from the group consisting of uridine, cytidine, glutamine, pantothenic acid, iron, colostrinin and caffeine.

### BACKGROUND OF THE INVENTION

The human brain has been estimated to contain 80 to 90 billion non-neuronal cells (glial cells) as well as 80 to 90 billion neurons. These cells pass signals to each other via as many as 1000 trillion synaptic connections.

The brain monitors and regulates the body's actions and reactions. It continuously receives sensory information, and rapidly analyses these data and then responds, controlling bodily actions and functions. The brainstem controls breathing, heart rate, and other autonomic processes that are independent of conscious brain functions. The neocortex is the centre of higher-order thinking, learning, and memory. The cerebellum is responsible for the body's balance, posture, and the coordination of movement.

Despite being protected by the thick bones of the skull, suspended in cerebrospinal fluid, and isolated from the bloodstream by the blood-brain barrier, the human brain is susceptible to many types of damage and disease in addition to damage due to normal aging.

There is a need for a food supplement that will help maintain the health and support the function of the brain, including cognitive function such as memory.

### SUMMARY OF THE INVENTION

The present invention relates to a food supplement to support brain function, including cognitive function such as memory. The food supplement comprises one or more active ingredients selected from the group consisting of uridine, cytidine, glutamine, pantothenic acid, iron, colostrinin and caffeine.

### DETAILED DESCRIPTION OF THE INVENTION

The present invention relates to a food supplement comprising an active ingredient selected from the group consisting of uridine, cytidine, glutamine, pantothenic acid and iron (hereafter ‘food supplement’ or ‘supplement’). The food supplement may further comprise other ingredients such as colostrinin, caffeine or vitamin B12.

The food supplement of the present invention is formulated to support healthy brain function. The food supplement is further designed to support an increase of activity in neurons and support the health of the brain, which in turns helps to slow down the ageing process.

The supplement may aid cognitive functions including perception and memory. The food supplement may also enhance concentration, alertness and learning. The food supplement has neuroprotective activity and may ameliorate or slow the rate of decline of cognitive function due to age, disease or accident. More particularly, the food supplement of the present invention may prevent, ameliorate or slow the rate of decline of memory loss due to age, disease or accident. The food supplement may further ameliorate or slow the rate of neurodegeneration due to age, disease or accident.

The food supplement may be useful in persons with damage due to one or more of the following:

Age-related memory impairment: Normal ageing is associated with a decline in various memory abilities in many cognitive tasks; this is known as age-related memory impairment (AMI) or age-associated memory impairment (AAMI). Studies comparing the effects of ageing on episodic memory, semantic memory, short-term memory and priming find that episodic memory is especially impaired in normal ageing; some types of short-term memory are also impaired. The deficits may be related to impairments seen in the ability to refresh recently processed information. Source information is one type of episodic memory that suffers with old age; this kind of knowledge includes where and when the person learned the information. Knowing the source and context of information can be extremely important in daily decision-making, so this is one way in which memory decline can affect the lives of the elderly.

Long-term illnesses, including epilepsy, Alzheimer's Dementia and mild cognitive impairment can also lead to memory loss.

Epilepsy as a brain activity occurs on a specific region or site of the brain, particularly the medial temporal lobe. Repeated attacks can damage brain tissues in some of the most important areas of the brain, such as the hippocampus which is essential to the processing of information and memory. Damage to the brain can cause problems in the storage of information and ultimately, in remembering them. Damage on the left side of the brain can lead to the loss of verbal memory. As a result, a person will find it difficult to remember words, whether they are spoken or written. If damage occurs on the right side of the brain, visual memory is affected. The person will find it difficult to remember what he saw and will have problems processing objects and directions. If damage is found on the frontal lobes, this can lead to short-term memory loss. When this happens, the person will find it difficult to keep their attention for long.

Dementia is the most serious form of memory problem. Dementia is a condition of the brain that causes a gradual loss of mental ability, including problems with memory, understanding, judgment thinking and language. In addition, other problems commonly develop such as changes in personality and changes in the way someone interacts with others in social situations. As dementia progresses, the ability of someone to look after themselves from day to day may also become affected.

Dementia is a brain malfunction that restricts a person's normal activities and generally results in the need for long-term care. As the dementia progresses, remembering, learning and communicating become difficult. Causes of dementia include head injury or trauma, stroke or a brain tumor, all of which destroy brain cells. Dementia also may run in families.

Alzheimer's disease is the most common form of dementia. Alzheimer's disease is a physical disease affecting the brain. During the course of the disease, ‘plaques’ and ‘tangles’ develop in the structure of the brain, leading to the death of brain cells.

Alzheimer's also leads to a shortage of some important chemicals in their brains. These chemicals are involved with the transmission of messages within the brain. Alzheimer's is a progressive disease, which means that gradually, over time, more parts of the brain are damaged. As this happens, the symptoms become more severe.

Mild cognitive impairment (MCI): Recently, some doctors have begun to use the term ‘mild cognitive impairment’ (MCI) when an individual has difficulty remembering things or thinking clearly but the symptoms are not severe enough to warrant the diagnosis of Alzheimer's disease. Recent research has shown that a small number of individuals with MCI have an increased risk of progressing to Alzheimer's disease. However, the conversion rate from MCI to Alzheimer's is small (about 10-15 per cent), and consequently a diagnosis of MCI does not always mean that the person will go on to develop Alzheimer's.

MCI is an intermediate stage between the expected cognitive decline of normal ageing and the more pronounced decline of dementia. It involves problems with memory, language, thinking and judgment that are greater than typical age-related changes. If you have mild cognitive impairment, you may be aware that your memory or mental function has “slipped.” And family and close friends may also notice a change. Generally these changes are not severe enough to interfere with day-to-day life and usual activities. Mild cognitive impairment increases your risk of later developing dementia, including Alzheimer's disease, especially when your main difficulty is with memory.

Symptoms that indicate possible MCI include: forgetting things more often; forgetting important events such as appointments or social engagements; loosing train of thought or the thread of conversations, books or movies; feeling increasingly overwhelmed by making decisions, planning steps to accomplish a task or interpreting instructions; having trouble finding the way around familiar environments; becoming more impulsive or show increasingly poor judgment.

Short or Sudden illnesses: Situations of sudden illness are often caused by an unexpected event, such as an accident or sudden change in the patient's health status.

Stroke can have a detrimental effect on short term memory. Long term memory is usually well preserved in people who have suffered a stroke. After a stroke a person will noticeably lose attention and concentration. Specific damage to the brain will aggravate this condition. As a result, the process of receiving and storing material may become faulty and information in the temporary store disappears.

Meningitis is defined as inflammation of the membranes that cover your spinal cord and brain. These protective membranes, called “meninges,” can become inflamed for a variety of reasons, but is considered a life threatening, medical emergency due to the fact that it can easily affect one's brain and spinal cord. Meningitis can also cause considerable memory loss, confusion, and the inability to concentrate and focus.

Physical trauma, such as a head injury in a car accident, can have effects on their memory. The most common form of memory disturbance in cases of severe injuries or perceived physical distress due to a traumatic event is post-traumatic stress disorder. Damage to different areas of the brain can have varied effects on memory. The temporal lobes, on the sides of the brain, contain the hippocampus and amygdala, and therefore have a lot to do with memory transition and formation. Patients who have had injury to this area have experienced problems creating new long-term memories. Common causes of minor to severe head injury resulting in brain impairment and memory loss include motor vehicle traffic collisions, home and occupational accidents, falls, assaults and bicycle accidents.

### Ingredients and Formulations

In one aspect the food supplement of the present invention comprises uridine. In another aspect the food supplement comprises cytidine. In another aspect the food supplement comprises glutamine. In another aspect the food supplement comprises pantothenic acid. In another aspect the food supplement comprises colostrinin or a fragment thereof. In another aspect the food supplement comprises elemental iron. In another aspect the food supplement comprises caffeine. In another aspect the food supplement comprises uridine and cytidine. In another aspect the food supplement comprises uridine, cytidine and glutamine. In another aspect the food supplement comprises uridine, cytidine and pantothenic acid. In another aspect the food supplement comprises uridine, cytidine and colostrinin. In another aspect the food supplement comprises uridine, cytidine, glutamine, and pantothenic acid. In another aspect the food supplement comprises uridine, cytidine, glutamine and colostrinin. In another aspect the food supplement comprises uridine, cytidine, colostrinin and pantothenic acid. In another aspect the food supplement comprises uridine, cytidine, glutamine, pantothenic acid and elemental iron. In another aspect the food supplement comprises uridine, cytidine, glutamine, pantothenic acid, elemental iron and caffeine. In another aspect the food supplement comprises uridine, cytidine, colostrinin and pantothenic acid glutamine, elemental iron and caffeine.

In another aspect the food supplement further comprises one or more additional ingredient selected from the group consisting of vitamin C, vitamin B12, vitamin B6, vitamin B complex, omega 3 fatty acid, phosphatidylserine, Ginkgo biloba extract, colostrum extract, evening primrose and folic acid.

The ingredients of the food supplement may be either in a neutral molecular form or in an ionized salt form.

In one embodiment the elemental iron is ferrous iron. In another embodiment the ferrous iron is selected from ferrous sulfate, ferrous fumarate, and ferrous gluconate. In another embodiment the food supplement comprises elemental iron and vitamin C. In another embodiment the uridine or cytidine is a phosphate salt.

In one embodiment the colostrum extract comprises colostrinin. In one embodiment the colostrinin is a nonapeptide of clostrinin designated NP having the amino acid sequence: Val-Glu-Ser-Tyr-Val-Pro-Leu-Phe-Pro. The term “colostrinin”, as used herein refers to a polypeptide which, in its natural form, is obtained from mammalian colostrum. Colostrinin is sometimes known as “colostrinine”, and has the following properties: (i) it has a molecular weight in the range 16,000 to 26,000 Daltons; (ii) it is a dimer or trimer of sub-units each sub-unit having a molecular weight in the range 5,000 to 10,000 Daltons, preferably 6,000 Daltons; (iii) it contains proline, and the amount of proline is greater than the amount of any other single amino acid (U.S. Pat. No. 6,852,700, incorporated by reference in its entirety).

In one embodiment the omega-3 fatty acid is docosahexaenoic acid (DHA). In another embodiment the omega-3 fatty acid is in the form of fish oil.

The specific dosage regimen and levels can be varied depending upon a variety of factors including the age, body weight, general health, sex, diet, time of administration, route of administration, etc. but each ingredient is present in amount that is safe.

The food supplement of the present invention may be added to or in the form of any suitable food or beverage. The food supplement of the present invention may also be in form of a tablet, capsule, powder, liquid drink or other form suitable for parenteral administration. A unit dose, the amount of food supplement consumed at one time, may further be divided into multiple tablets, capsules, etc. or divided into different forms, e.g., a food and a beverage.

In other embodiments the ingredients, either separately or combined, are within the following unit or daily dose ranges:

Uridine—between about 50 mg and about 500 mg per unit dose or between about 50 mg and about 1 g per day.

Cytidine—between about 50 mg and about 500 mg per unit dose or between about 50 mg and about 1 g per day;

Glutamine—between about 15 and about 500 mg per unit dose or between about 50 mg to about 1 g per day;

Elemental iron—between about 1 mg and about 120 mg per unit dose or per day;

Pantothenic acid—between about 0.5 mg and about 1 g per unit dose or per day.

In some embodiments the ingredients, either separately or combined, are with the following unit or daily dose ranges:

Uridine—between about 50 mg and about 350 mg per unit dose or between about 50 mg and about 500 mg per day;

Cytidine—between about 50 mg and about 350 mg per unit dose or between about 50 mg and about 500 mg per day;

Glutamine—between about 15 mg and about 350 mg per unit dose or between about 15 mg to about 500 mg per day;

Elemental iron—between about 1 mg and about 60 mg per unit dose or per day;

Pantothenic acid—between about 0.5 mg and about 100 mg per unit dose or per day.

In some embodiments the ingredients, either separately or combined, are with the following unit or daily dose ranges:

Uridine—between about 75 mg and about 150 mg per unit dose or between about 150 mg and about 300 mg per day;

Cytidine—between about 75 mg and about 150 mg per unit dose or between about 150 mg and about 300 mg per day;

Glutamine—between about 25 mg and about 50 mg per unit dose or between about 50 mg to about 100 mg per day;

Elemental iron—between about 1 mg and about 10 mg per unit dose or per day;

Pantothenic acid—between about 0.5 mg and about 10 mg per unit dose or per day.

In some embodiments, the amount of caffeine per unit dose is between about 10 mg to about 250 mg, between about 10 mg to about 100 mg, or between about 10 mg to about 50 mg.

In some embodiments, a unit dose of the food supplements comprises an amount of an ingredient selected from the group consisting of: about 150 mg uridine, about 150 mg cytidine, about 50 mg glutamine, about 2 mg elemental iron and about 1 mg pantothenic acid. In some embodiments, a unit dose of the food supplements comprises about 150mg uridine, about 150 mg cytidine, about 50 mg glutamine, about 2.1 mg elemental iron, and about 0.9 mg pantothenic acid.

In one aspect the formulation comprises an antioxidant. In another aspect the formulation comprises an antioxidant selected from the group consisting of ascorbic acid (vitamin C), sodium ascorbate, calcium ascorbate, potassium ascorbate, fatty acid esters of ascorbic acid (ascorbyl palmitate), ascorbyl stearate, Vitamin E, tocotrienols, alpha-tocotrienol, beta-tocotrienol, gamma-tocotrienol, delta-tocotrienol, tocopherols (natural), alpha-tocopherol, beta-tocopherol, gamma-tocopherol, delta-tocopherol, propyl gallate, octyl gallate, dodecyl gallate, ethyl gallate, guaiac resin, erythorbic acid, sodium erythorbate, erythorbin acid, sodium erythorbin, tert-butylhydroquinone (TBHQ), butylated hydroxyanisole (BHA), butylated hydroxytoluene (BHT), anoxomer, ethoxyquin, sodium phosphates (i) monosodium phosphate (ii) disodium phosphate (iii)trisodium phosphate, potassium phosphates (i) monopotassium phosphate (ii) dipotassium phosphate (iii) tripotassium phosphate.

In one aspect the formulation comprises an antioxidant in the form of a fruit extract. In some embodiments the extract is selected from the group consisting of blueberry, black raspberry, pomegranate, red raspberry, strawberry, black plum, cherry, red grape, goji berry, acai berry, and fig extract. In some embodiments the extract is selected from blueberry, black raspberry, pomegranate, goji berry or acai berry extract.

In one embodiment the unit dose is administered once daily. In one embodiment the unit dose is administered twice daily.

The food supplement and dosage forms described herein can be administered via any conventional route of administration. In one embodiment the route of administration is oral. Examples of suitable oral compositions of the present invention include tablets, capsules, troches, lozenges, suspensions, dispersible powders or granules, emulsions, syrups and elixirs.

The food supplement may be administered as a formulation in association with one or more additional ingredients or excipients. The term “excipient” refers to an inactive substance used as a carrier for the active ingredient(s) and includes antiadherents, binders, coatings, disintegrants, fillers and diluents, flavours, colours, glidants, dispersing agents, wetting agents, lubricants, preservatives, sorbents and sweeteners. The choice of excipient will to a large extent depend on factors such as the particular mode of administration, the effect of the excipient on solubility and stability, and the nature of the dosage form.

Examples of fillers and diluents of the present invention include, for example, calcium carbonate, sodium carbonate, lactose, calcium phosphate, sodium phosphate and plant cellulose (pure plant filler).

A range of vegetable fats and oils may be used in soft gelatin capsules. Other examples of fillers of the present invention include sucrose, glucose, mannitol, sorbitol, and magnesium stearate.

Examples of granulating and disintegrants of the present invention include corn starch and alginic acid, crosslinked polyvinyl pyrrolidone, sodium starch glycolate or crosslinked sodium carboxymethyl cellulose (crosscarmellose).

Examples of binding agents of the present invention include starch, gelatin, acacia, cellulose, cellulose derivatives, such as methyl cellulose, microcrystalline cellulose and hydroxypropyl cellulose, polyvinylpyrrolidone, sucrose, polyethylene glycol, lactose, or sugar alcohols like xylitol, sorbitol and maltitol.

Examples of lubricants of the present invention include magnesium stearate, calcium stearate, stearic acid and talc.

Examples of coatings of the present invention include a hydroxy propylmethylcellulose (HPMC) and gelatin.

Tablets of the present invention may be uncoated or coated by known techniques. Such coatings may delay disintegration and thus, absorption in the gastrointestinal tract and thereby provide a sustained action over a longer period.

Hard gelatin capsules constitute another solid dosage form of the present invention for oral use. Such capsules similarly include the active ingredients mixed with carrier materials as described above. Soft gelatin capsules include the active ingredients mixed with water-miscible solvents such as propylene glycol, PEG and ethanol, or an oil such as peanut oil, liquid paraffin or olive oil.

Aqueous suspensions are also contemplated as containing the active material in admixture with excipients suitable for the manufacture of aqueous suspensions. Such excipients include suspending agents, for example sodium carboxymethylcellulose, methylcellulose, hydroxypropylmethylcellulose, sodium alginate, polyvinylpyrrolidone, tragacanth and acacia; dispersing or wetting agents, e.g., lecithin; preservatives, e.g., ethyl, or n-propyl para-hydroxybenzoate, colorants, flavors, sweeteners and the like.

Dispersible powders and granules suitable for preparation of an aqueous suspension by the addition of water provide the active ingredients in admixture with a dispersing or wetting agent, suspending agent and one or more preservatives. Suitable dispersing or wetting agents and suspending agents are exemplified by those already mentioned above. Syrups and elixirs may also be formulated.

1. A composition comprising a food supplement comprising one or more ingredients selected from the group consisting of uridine, cytidine, glutamine, pantothenic acid, iron, colostrinin and caffeine.

2. The composition of claim 1, wherein said food supplement further comprises one or more ingredients selected from the group consisting of vitamin C, vitamin B 12, vitamin B6, vitamin B complex, omega 3 fatty acid, phosphatidylserine, Ginkgo biloba extract, evening primrose and folic acid.

3. The composition of claim 1, wherein said food supplement comprises one or more of ingredient in an amount selected from the group consisting of: between about 50 mg and about 1 g per unit dose of uridine; between about 50 mg and about 1 g per unit dose of cytidine; between about 15 and about 1 g per unit dose of glutamine; between about 1 mg and about 120 mg per unit dose of elemental iron; and between about 0.5 mg and about 1 g per unit dose of pantothenic acid.

4. The composition of claim, wherein said food supplement comprises one or more of ingredient in an amount selected from the group consisting of: between about 50 mg and about 500 mg per unit dose of uridine; between about 50 mg and about 500 mg per unit dose of cytidine; between about 15 and about 500 mg per unit dose of glutamine; between about 1 mg and about 60 mg per unit dose of elemental iron; between about 0.5 mg and about 500 mg per unit dose of pantothenic acid.

5. The composition of claim 1, wherein said food supplement comprises one or more of ingredient in an amount selected from the group consisting of: between about 50 mg and about 250 mg per unit dose of uridine; between about 50 mg and about 250 mg per unit dose of cytidine; between about 15 and about 150 mg per unit dose of glutamine; between about 1 mg and about 10 mg per unit dose of elemental iron; between about 0. 5 mg and about 10 mg per unit dose of pantothenic acid.

6. The composition of claim 1, wherein a unit dose of said food supplement comprises one or more ingredient in an amount selected from the group consisting of: about 150 mg uridine, about 150 mg cytidine, about 50 mg glutamine, about 2 mg elemental iron and about 1 mg pantothenic acid.

7. The composition of claim 1, wherein a unit dose of said food supplement comprises one or more ingredient in an amount selected from the group consisting of: about 150 mg uridine, about 150 mg cytidine, about 50 mg glutamine, about 2.1 mg elemental iron, and about 0.9 mg pantothenic acid.

8. The composition of claim 1, wherein a unit dose of said food supplement comprises between about 10 mg to about 250 mg, between about 10 mg to about 100 mg, or between about 10 mg to about 50 mg of caffeine.

9. The composition of claim 1, wherein said food supplement comprises uridine and cytidine.

10. The composition of claim 1, wherein said food supplement comprises glutamine.

11. The composition of claim 1, wherein said food supplement comprises pantothenic acid.

12. The composition of claim 1, wherein said food supplement comprises iron.

13. The composition of claim 1, wherein said food supplement comprises colostrinin.

14. The composition of claim 1, wherein said food supplement comprises caffeine.

15. The composition of claim 1, wherein said food supplement comprises an antioxidant.

16. The composition of claim 1, wherein a unit dose of the food supplement is in the form of a liquid, powder, tablet or capsule.

17. The composition of claim 16, wherein said unit dose is one or two capsules.

18. The composition of claim 17, wherein each of said one or two capsules comprises about 75 mg uridine, about 75 mg cytidine, about 25 mg glutamine, about 0.45 mg pantothenic acid and about 1.05 mg iron.

19. The composition of claim 16, wherein said powder comprises about 75 mg uridine, about 75 mg cytidine, about 25 mg glutamine, about 0.45 mg pantothenic acid and about 1.05 mg iron.

20. The composition of claim 16, wherein said powder comprises about 150 mg uridine, about 150 mg cytidine, about 50 mg glutamine, about 0.9 mg pantothenic acid and about 2.1 mg iron.

21. A method comprising administration of the food supplement of claim 1 to an individual for supporting healthy brain function.

22. The method of claim 21, wherein said brain function is cognitive function.

23. The method of claim 22, wherein said cognitive function is memory.
